(19) United States
(12) Patent Application Publication
(10) Pub. No.: US 2020/0214898 A1
(43) Pub. Date: Jul. 9, 2020

(54) ABSORBENT WOUND DRESSING THAT INCORPORATES A NOVEL WOUND FLUID INDICATING SYSTEM

(71) Applicants: KCI USA, INC., San Antonio, TX (US); SYSTAGENIX WOUND MANAGEMENT, LIMITED, West Sussex (GB)

(72) Inventors: Alexander WAITE, Cowling (GB); Katherine A. BOURDILLON, Leeds (GB)

(21) Appl. No.: 16/624,437

(22) PCT Filed: Jun. 19, 2018
(86) PCT No.: PCT/US2018/038277

§ 371 Date: Dec. 19, 2019

Related U.S. Application Data
(60) Provisional application No. 62/524,975, filed on Jun. 26, 2017.

(51) Int. Cl. A61F 13/00; A61F 13/02

(52) U.S. Cl. CPC A61F 13/00055; A61F 13/00029; A61F 2013/00846; A61F 13/00042; A61F 2013/00944; A61F 13/0206

(57) ABSTRACT

The wound dressing described herein includes a backing layer, absorbent layer, and foam layer. The wound dressing also includes one or more visual indicators. The visual indicators are substantially non-visible in their initial, dehydrated state. As the visual indicators absorb fluid they swell and become visible. The visual indicators enable a caregiver to know when the wound dressing has reached, or is about to reach, its fluid capacity.

### CROSS-REFERENCE TO RELATED APPLICATIONS

This application claims the benefit of priority to U.S. Provisional Application No. 62/524,975, filed on Jun. 26, 2017, which is incorporated herein by reference in its entirety.

### BACKGROUND OF THE DISCLOSURE

Maintaining a moist wound environment can promote the healing of wounds, especially burns and chronic wounds such as ulcers. Wound fluid is produced by the wound during the healing process. Wound fluid is mainly composed of water, but also contains nutritional requirements, proteins, and cells. Wound fluid represents an essential component of the healing process. However, in the case of chronic wounds prolonged inflammation and infection can lead to excess and prolonged production of exudate. Absorbent dressings are designed to manage the excess exudate produced. Absorbent wound dressings can have a maximum fluid handling capacity. Once breached, the dressing can fail and the wound fluid can escape the confines of the wound area. This can cause patient discomfort and can cause maceration of skin adjacent to the wound. Predicting or otherwise determining when a wound dressing has reached its maximum fluid handling capacity can be difficult.

### SUMMARY OF THE DISCLOSURE

The disclosure describes an absorbent wound dressing that incorporates a staged visual indicator of the wound dressing's saturation level. The visual indicator enables the caregiver to track wound fluid production rates and more accurately predict when a wound dressing change is needed. The visual indicator also provides an early warning for dressing failure.

According to one aspect of the disclosure, a wound dressing can include a backing layer. The backing layer can include a first environment-facing side and a first wound-facing side. The backing layer can be liquid impermeable and vapor permeable. The wound dressing can include a foam layer. The foam layer can include a second wound-facing side and a second environment-facing side that is coupled with the first wound-facing side of the backing layer. The wound dressing can include an absorbent layer that is positioned between the backing layer and the foam layer. The absorbent layer can include a third wound-facing side and a third environment-facing side. The wound dressing can include a fluid indicator positioned between the backing layer and the foam layer. The fluid indicator can be configured to transition from an initial state to a swollen state in response to absorbing a predetermined amount of liquid.

In some implementations, at least one fluid indicator can be substantially non-visible in the initial state and substantially visible in the swollen state. The wound dressing can include a plurality of fluid indicators positioned between the backing layer and the foam layer. The plurality of fluid indicators positioned between the backing layer and the foam layer can be in a pattern that radiates outward from a central position of the absorbent layer. A first portion of the plurality of fluid indicators can include a first color pigment and a second portion of the plurality of fluid indicators can include a second color pigment. The second color pigment can be different than the first color pigment. The color intensity of each of the plurality of fluid indicators can increase with respect to a distance from a central position of the absorbent layer.

The plurality of fluid indicators can radiate or extend outward from a perimeter of the absorbent layer. The fluid indicator can include sodium polyacrylate (SAP). The fluid indicator can have a substantially spherical shape. The fluid indicator can have a substantially ring shape and encircle a perimeter of the absorbent layer. The fluid indicator can be a visual fluid indicator. The backing layer can include a substantially transparent portion. The fluid indicator, in the swollen state, can be visible through the substantially transparent portion. The fluid indicator can be a tactile fluid indicator.

The backing layer can include a stretchable portion that is configured to deform when the fluid indicator is in the swollen state. The size of the fluid indicator in the initial state is between about 0.1 mm and about 3 mm in diameter. The size of the fluid indicator in the swollen state is between about 1 mm and about 10 mm in diameter. The backing layer can include a polyurethane film. The wound dressing can include an adhesive layer that is coupled with the first wound-facing side of the backing layer. The foam layer can include a hydropolymer foam. The absorbent layer can include a wicking material that is configured to distribute fluid through the absorbent layer.

The foregoing general description and following description of the drawings and detailed description are exemplary and explanatory and are intended to provide further explanation of the invention as claimed. Other objects, advantages, and novel features will be readily apparent to those skilled in the art from the following brief description of the drawings and detailed description.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 illustrates an example wound dressing with hydrated visual indicators.

FIG. 2 illustrates an example wound dressing with dehydrated visual indicators.

FIG. 3 illustrates an exploded view of the example wound dressing illustrated in FIG. 1.

FIGS. 4 and 5 illustrate a cross-sectional view of the example wound dressing illustrated in FIG. 1.

FIG. 6 illustrates an example wound dressing with ring-shaped visual indicators.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Presently, due to the lack of an accurate way to predict fluid production rates, wound dressings can fail when they reach their maximum fluid handling capacity. When wound fluid production exceeds the fluid handling capacity of the wound dressings and moves outside the confines of the wound dressing, the fluid can cause additional tissue damage in the form of maceration. The wound dressing described herein includes visual indicators that can assist in determining when a wound dressing has reached, or is about to reach, its maximum fluid handling capacity. The visual indicator can alert a caregiver or wearer that the wound dressing should be replaced before the wound dressing reaches its maximum fluid handling capacity by providing an early warning for dressing failure. The caregiver can also use the visual indicators to track the rate of wound fluid production.

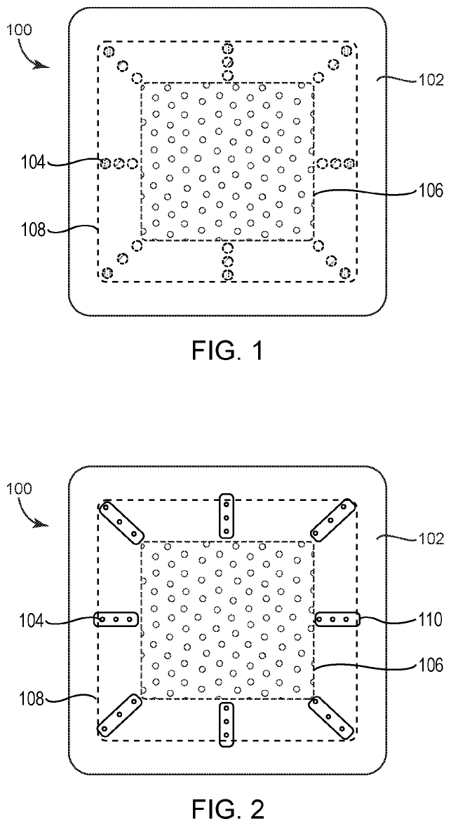

FIG. 1 illustrates an example wound dressing 100. The wound dressing 100 includes a top, backing layer 102. Beneath the backing layer 102, as indicated by the dotted lines, the wound dressing 100 includes an absorbent layer 106 and a foam layer 108. The wound dressing 100 also includes several fluid indicators 104. The indicators can be referred to as visual fluid indicators 104 and tactile fluid indicators 104 and are discussed below in greater detail.

The wound dressing's fluid indicators 104 provide a visual or tactile indication as to the amount of fluid contained within the wound dressing 100. The indicators 104 can be spherical beads positioned between the foam layer 108 and the backing layer 102. The fluid indicators 104 can transition from an initial state to a swollen state as they absorb fluid. As the fluid indicators 104 swell they transition from a substantially non-visible state to a substantially visible state. The fluid indicators 104 can be colored to make their visualization more prominent as the fluid indicators 104 swell.

The fluid indicators 104 can be sodium polyacryalte (SAP) beads. The foam layer 108 absorbs the wound fluid produced by the wound. The absorbent layer 106 can wick the fluid from the foam layer 108. As the fluid saturates the absorbent layer 106 and/or the foam layer 108, the fluid can migrate to, and be absorbed by, the fluid indicators 104. As the fluid indicators 104 absorb the fluid, the fluid hydrates the fluid indicators 104 and the fluid indicators 104 become visible.

In some implementations, the fluid indicators 104 are located in a set pattern that radiates outwardly from the center of the wound dressing 100 (or from the perimeter of the absorbent layer 106). The fluid indicators 104 can have different colors that can indicate a distance from the center of the wound dressing 100. For example, the fluid indicators 104 closest to the center of the wound dressing 100 can be green, the fluid indicators 104 an intermediate distance from the center can be yellow, and the fluid indicators 104 the greatest distance from the center of the wound dressing 100 can be red indicating a progression of wound fluid to a stage where replacement of the dressing is appropriate.

The fluid indicators 104 enable the caregiver to determine the wound dressing's relative remaining fluid capacity. For example, when the red fluid indicators 104 are visible, the wound dressing 100 has relatively little remaining fluid capacity and the wound dressing 100 should be replaced shortly. By monitoring the time interval between placement of the wound dressing 100 and when each of the fluid indicators 104 become visible, the caregiver can also monitor and track the wound's fluid relative production rate. By measuring the fluid production rate, the caregiver can also predict when the wound dressing 100 should be replaced.

In some implementations, the fluid indicators 104 have a non-hydrated diameter between about 0.1 mm and about 5 mm, between about 0.1 mm and about 3 mm, or between about 0.1 mm and about 2 mm. The fluid indicators 104 can have a hydrated (or swollen) diameter between about 1 mm and about 10 mm, between about 1 mm and about 8 mm, between about 1 mm and about 6 mm, or between about 1 and about 3 mm.

The wound dressing's backing layer 102 is a liquid impermeable and vapor permeable barrier. The backing layer 102 can be impermeable to wound exudate or other wound fluids. The backing layer 102 provides a barrier to the passage of microorganisms, bacteria, and other contaminants through the wound dressing 100. The backing layer 102 enables the other components of the wound dressing 100 to retain fluid to promote a moist wound environment.

The backing layer 102 can be a thin layer of polyurethane film. For example, the backing layer 102 can include the polyurethane film ESTANE 5714F. The backing layer 102 can include poly alkoxyalkyl acrylates and methacrylates. In some implementations, the backing layer 102 includes a continuous layer of a high-density blocked polyurethane foam that is predominantly closed-cell. The backing layer 102 can have a thickness between about 10 μm and about 100 μm, between about 25 μm and about 75 μm, or between about 50 μm and about 75 μm.

The backing layer 102 extends beyond the perimeter of the absorbent layer 106 and the foam layer 108. The backing layer's wound-facing side can be coated with an acrylic or other adhesive. The adhesive couples with the absorbent layer 106 and the foam layer 108. The portion of the backing layer 102 extending past the foam layer 108 adheres to the patient or other surface during wear time. The backing layer's adhesive-coated margin that extends past the foam layer 108 can extend past all sides of the foam layer 108 such that wound dressing 100 is a so-called island dressing. In other embodiments, the adhesive-coated margin can be eliminated and wound dressing 100 can be adhered to a surface using other techniques.

The adhesive applied to the wound-facing side of the backing layer 102 can be moisture vapor transmitting. The application of the adhesive can be patterned to enable the passage of water vapor through the backing layer 102. The adhesive may include a continuous moisture vapor transmitting, pressure-sensitive adhesive layer of the type, conventionally used for island-type wound dressings (e.g., a polyurethane-based pressure sensitive adhesive). One example of an adhesive which can be used is a pressure sensitive adhesive based on acrylate ester copolymers, polyvinyl ethyl ether and polyurethane.

The wound dressing 100 also includes the absorbent layer 106. The absorbent layer 106 is configured to wick moisture from the foam layer 108 and distribute the moisture through the absorbent layer 106. In some implementations, the absorbent layer 106 can include a hydrogel or hydrogel composition. Several examples of hydrogels and hydrogel compositions which can be used to the absorbent layer 106 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein.

The expressions “hydrogel” and “hydrogel compositions” can include any hydrophilic gels and gel compositions. The compositions can include organic non-polymeric components in the absence of water. For example, the absorbent layer 106 can be formed from a polyurethane that entraps water to form a gel. The absorbent layer 106 can be substantially continuous, substantially non-porous, or non-foamed. The absorbent layer 106 can include a flexible plasticized hydrophilic polymer matrix having a substantially continuous internal structure. The density of absorbent layer 106 may be between about 0.5 g/cm³ and about 1.1 g/cm³, between about 0.8 g/cm³ and about 1.1 g/cm³, or between about 0.9 and about 1.1 g/cm³. The thickness of the absorbent layer 106 can be between about 1 mm and about 10 mm, between about 2 mm and about 7 mm, or between about 2 mm and about 5 mm.

In some implementations, the absorbent layer 106 is cross-linked. The absorbent layer 106 can be substantially insoluble in water at ambient temperatures. The absorbent layer 106 can absorb and entrap liquid to provide a highly hydrated gel structure in contrast to the porous foam structure of foam layer 108. The gel of the absorbent layer 106 can absorb between about 1 g/g and about 10 g/g, between about 2 g/g and about 7 g/g, or between about 2 g/g and about 5 g/g of physiological saline at 200.

In some implementations, the dry weight of the absorbent layer 106 is from about 1000 g/m² to about 5000 g/m² or between about 2000 g/m² to about 4000 g/m². In some implementations, the absorbent layer 106 includes between about 1% and about 30%, between about 5% and about 25%, or between about 10% and about 20% by weight of water before use. The absorbent layer 106 can contain between about 1% and about 40%, between about 5% and about 20%, or between about 5% and about 15% by weight one or more humectants. The humectants can include glycerol, propylene glycol, sorbitol, mannitol, polydextrose, sodium pyrrolidine carboxylic acid (NaPCA), hyaluronic acid, aloe, jojoba, lactic acid, urea, gelatin, lecithin, or any combination thereof. The entrapped water and optional humectants can give the hydrogel a soft, moist wound-friendly surface for contacting the wound.

The wound dressing 100 also includes the foam layer 108. The foam layer 108 can be a hydrophilic foam layer. The foam layer 108 may include a polyurethane foam. In some implementations, the foam layer 108 includes a flexible plasticized hydrophilic polymer matrix having an internal cellular structure. Several examples of hydrophilic foams which can be used to form the foam layer 108 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein.

The foam layer 108 can provide enhanced absorbency for liquid exudate. This is because the initial substantially anhydrous condition and porous structure of the foam layer 108 enables it to absorb a larger amount of water by both chemical and physical absorption that is the case for the corresponding hydrogel material. Furthermore, the porous structure of the foam provides for rapid uptake of liquid exudate, in contrast to pure hydrogel dressings.

In some implementations, the foam layer 108 has a thickness between about 1 mm and about 20 mm, between about 1 mm and about 10 mm, or between about 1.5 mm and about 5 mm. In some implementations, the foam layer 108 has a density between about 0.28 g/cm³ and about 0.5 g/cm³ or between about 0.32 g/cm³ and about 0.48 g/cm³. The foam that forms the foam layer 108 may be hydrophilic and can absorb aqueous fluids such as wound exudate with swelling. The foam layer 108 may be highly cross-linked and substantially insoluble in water.

In some implementations, the foam layer 108 has an absorbency of at least 3 grams of saline per gram of foam. The foam layer 108 can have a swellability in water of at least 200%. In some implementations, the foam layer 108 is constructed using the foam as described in European Patent No. 0541391 issued Jun. 10, 1998, the entire disclosure of which is incorporated by reference herein. In some implementations, the foam layer 108 includes less than 10%, less than 5%, or less than 2% of water before use.

FIG. 2 illustrates an example wound dressing 100 prior to the absorption of fluid. In some implementations, as illustrated in FIG. 2, prior to the absorption of fluid, the fluid indicators 104 are not substantially visible. The wound dressing 100 illustrated in FIG. 2 also illustrates that the backing layer 102 can include windows 110 through which the fluid indicators 104 are visible or become visible as they swell. The windows 110 can be substantially clear or transparent portions of the backing layer 102.

The size of wound dressing 100 can vary depending on the size of the wound to be dressed. For example, it is contemplated that the size of wound dressing 100 can range from 1 cm² to 200 cm², and more preferably from 4 cm² to 100 cm². However, other shapes and sizes of wound dressing 100 are also possible depending on the intended use.

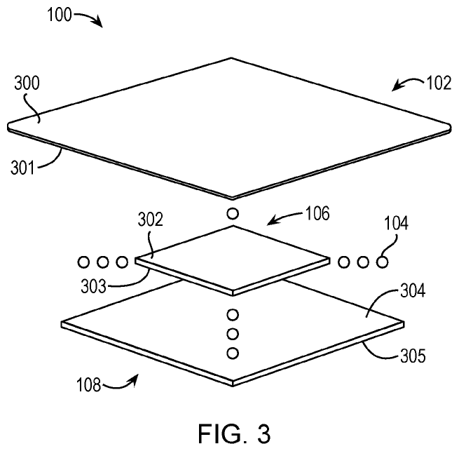

FIG. 3 illustrates an exploded view of an example wound dressing 100. The absorbent layer 106 and the fluid indicators 104 are positioned between the foam layer 108 and the backing layer 102. The backing layer 102 includes an environment-facing side 300 and a wound-facing side 301. The absorbent layer 106 includes an environment-facing side 302 and a wound-facing side 303. The foam layer 108 includes an environment-facing side 304 and a wound-facing side 305.

The backing layer 102 includes the environment-facing side 300 and the wound-facing side 301. The wound-facing side 301 can be coated with an adhesive. The adhesive can coat the wound-facing side 301 or can be patterned onto the wound-facing side 301. For example, the adhesive can be patterned on the margin of the wound-facing side 301 that is beyond the perimeter of the foam layer 108 and ultimately contacts that patient's skin.

The wound-facing side 301 of the backing layer 102 is coupled with the environment-facing side 302 of the absorbent layer 106. In some implementations, an adhesive can be used to couple the backing layer 102 and the absorbent layer 106 together. In other implementations, the coupling of the foam layer 108 and the backing layer 102 can seal the absorbent layer 106 between the foam layer 108 and the backing layer 102 and hold the absorbent layer 106 in position. Similarly, fluid indicators 104 can also be sealed between the backing layer 102 and foam layer 108 to be held in position. In other implementations, the fluid indicators 104 can be coupled with the environment-facing side 304 of the foam layer 108 or the wound-facing side 301 of the backing layer 102 prior to assembly of the backing layer 102, the absorbent layer 106, and the foam layer 108 into a wound dressing 100. In another configuration, the foam layer 108, absorbent layer 106, and fluid indicators 104 (or any combination thereof, hereinafter “island material”) are coupled to the backing layer 102 by a wound contact layer. For example, the wound contact layer is a perforated sheet (such as polyurethane film) coated on the wound-facing side with silicone, and the environment-facing side with a pressure-sensitive adhesive (such as acrylic adhesive). The wound contact layer is the same or substantially the same size as the backing layer 102, and is affixed to backing layer 102 via the pressure-sensitive adhesive, resulting in the island material being held in place between the wound contact layer and backing 102 (without the need for an adhesive between the island material and the backing layer 102).

In some implementations, the foam layer 108 is laminated to the absorbent layer 106 using a fusible fiber positioned between the absorbent layer 106 and the foam layer 108. For example, the absorbent layer 106 may be applied to environment-facing side 304 of the foam layer 108 and may at least partially cover the environment-facing side 304. The absorbent layer 106 can be bonded to the foam layer 108, for example by an adhesive or by radiation cross-linking. In some implementations, the absorbent layer 106 is bonded to the foam layer 108 by urethane or urea linkages. This can be achieved by applying the foam layer 108 to the absorbent layer 106 (substantially without mixing) before polyurethane curing is complete.

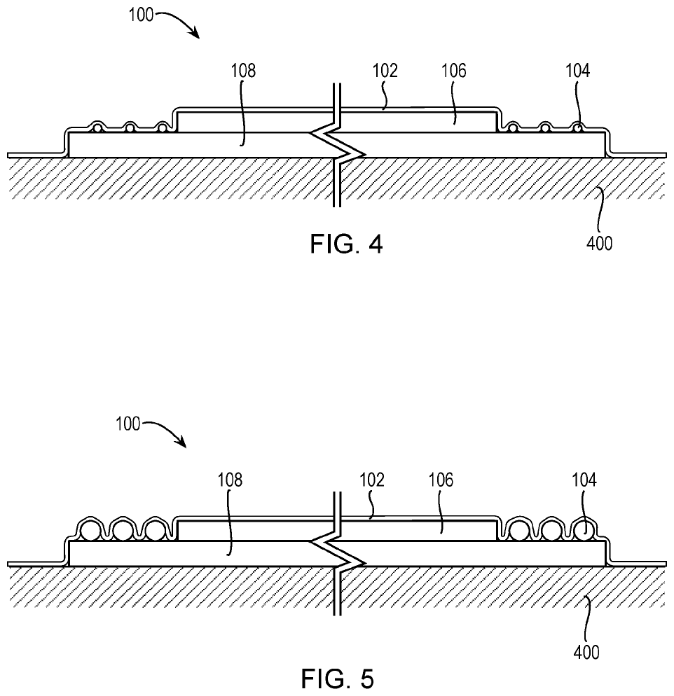

FIGS. 4 and 5 illustrate a cross-sectional view of the example wound dressing 100. FIG. 4 illustrates the fluid indicators 104 in their initial, dehydrated state. FIG. 5 illustrates the fluid indicators 104 in their hydrated, swollen state. The wound dressing 100 is coupled with the surface 400, which, for example, can be the patient's skin. Closest to the surface 400 is the foam layer 108. The fluid indicators 104 and the absorbent layer 106 sit atop the foam layer 108. The backing layer 102 provides a barrier that covers the other layers and secures the wound dressing 100 to the surface 400.

As illustrated in FIG. 4, in their initial, dehydrated state, the fluid indicators 104 are small and not easily visible through the backing layer 102. Once hydrated and swollen, as illustrated in FIG. 5, the fluid indicators 104 become visible. The backing layer 102 is configured to stretch to enable the fluid indicators 104 to expand towards the environment-facing side of the wound dressing 100 and away from the wound. In some implementations, the backing layer 102 can become less opaque in the stretched areas, making the fluid indicators 104 visible. As described above, the backing layer 102 can include windows 110 through which the fluid indicators 104 are visible.

As described above, the fluid indicators 104 can be colored. As the fluid indicators 104 swell, the coloring of the fluid indicators 104 can become visible through the backing layer 102. The fluid indicators 104 can be color coded to indicate their proximity to the center of the wound dressing 100. For example, the fluid indicator 104 closest to the absorbent layer 106 can be green colored, the next fluid indicator 104 can be yellow, and the fluid indicator 104 farthest from the absorbent layer 106 can be red.

In some implementations, the fluid indicators 104 can be tactile indicators of the wound dressing's current fluid absorption status. Also, as illustrated in FIG. 5, when the fluid indicators 104 swell they extend away from the surface 400 and create mounds, undulations, or other protrusions in the backing layer 102. The protrusions created by the swollen fluid indicators 104 can be felt when a caregiver or wearer runs their fingers across the surface of the backing layer 102. Detecting the wound dressing's current status with a tactile indicator can be advantageous when the wound dressing 100 is not easily viewed visually. For example, the wound dressing 100 may be placed on the back of the patient. The patient may not be able to visualize the wound dressing 100, but the patient is able to reach and touch the surface of the backing layer 102 to determine if the wound dressing 100 should be replaced. For example, feeling one of the three indicators 104 in an area the patient can determine the wound dressing 100 has not yet reached its fluid capacity. Later, feeling three of the three indicators 104 in an area, the patient can determine the wound dressing 100 should be changed shortly. In some implementations, the protrusions caused by the fluid indicators 104 are large enough to also be visualized.

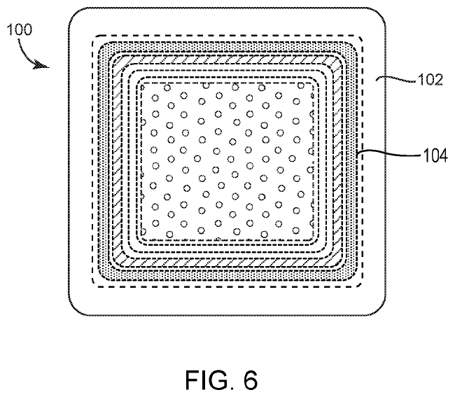

FIG. 6 illustrates another example wound dressing 100. The fluid indicators 104 can be formed into additional shapes other than beads. FIG. 6 illustrates an example wound dressing 100 where the fluid indicators 104 are ring shaped. As with the bead-shaped fluid indicators 104, the ring-shaped fluid indicators 104 can be color coded. For example, the inner most ring can be colored green, the middle ring can be colored yellow, and the outer most ring can be colored red.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

As used herein, the term “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. A wound dressing comprising:
a backing layer comprising a first environment-facing side, and a first wound-facing side, wherein said backing layer is liquid impermeable and vapor permeable;
a foam layer comprising a second wound-facing side and a second environment-facing side coupled with the first wound-facing side of the backing layer;
an absorbent layer positioned between the backing layer and the foam layer, the absorbent layer comprising a third wound-facing side and a third environment-facing side; and
a fluid indicator positioned between the backing layer and the foam layer, the fluid indicator configured to transition from an initial state to a swollen state in response to absorbing a predetermined amount of liquid.

2. The wound dressing of claim 1, wherein the at least one fluid indicator is substantially non-visible in the initial state and substantially visible in the swollen state.

3. The wound dressing of claim 1, further comprising a plurality of fluid indicators positioned between the backing layer and the foam layer.

4. The wound dressing of claim 3, wherein the plurality of fluid indicators positioned between the backing layer and the foam layer are in a pattern radiating outward from a central position of the absorbent layer.

5. The wound dressing of claim 3, wherein a first portion of the plurality of fluid indicators comprise a first color pigment and a second portion of the plurality of fluid indicators comprise a second color pigment different than the first color pigment.

6. The wound dressing of claim 3, wherein a color intensity of each of the plurality of fluid indicators increases with respect to a distance from a central position of the absorbent layer.

7. The wound dressing of claim 3, wherein the plurality of fluid indicators radiate outward from a perimeter of the absorbent layer.

8. The wound dressing of claim 1, wherein the fluid indicator comprises sodium polyacrylate (SAP).

9. The wound dressing of claim 1, wherein the fluid indicator has a substantially spherical shape.

10. The wound dressing of claim 1, wherein the fluid indicator has a substantially ring shape and encircles a perimeter of the absorbent layer.

11. The wound dressing of claim 1, wherein the fluid indicator is a visual fluid indicator.

12. The wound dressing of claim 11, wherein the backing layer comprises a substantially transparent portion and the fluid indicator in the swollen state is visible through the substantially transparent portion.

13. The wound dressing of claim 1, wherein the fluid indicator is a tactile fluid indicator.

14. The wound dressing of claim 13, wherein the backing layer comprises a stretchable portion configured to deform when the fluid indicator is in the swollen state.

15. The wound dressing of claim 1, wherein the size of the fluid indicator in the initial state is between about 0.1 mm and about 3 mm in diameter.

16. The wound dressing of claim 1, wherein the size of the fluid indicator in the swollen state is between about 1 mm and about 10 mm in diameter.

17. The wound dressing of claim 1, wherein the backing layer comprises a polyurethane film.

18. The wound dressing of claim 1, wherein further comprising an adhesive layer coupled with the first wound-facing side of the backing layer.

19. The wound dressing of claim 1, wherein the foam layer comprises a hydropolymer foam.

20. The wound dressing of claim 1, wherein absorbent layer comprises a wicking material configured to distribute fluid through the absorbent layer.
